(19) United States
(12) Patent Application Publication
(10) Pub. No.: US 2026/0158481 A1
(43) Pub. Date: Jun. 11, 2026

(54) CYCLIC BIARYL PHOSPHINES AS LIGANDS FOR PALLADIUM-CONTAINING CATALYSTS IN CROSS-COUPLING REACTIONS

(71) Applicant: HERAEUS PRECIOUS METALS GMBH & CO. KG, Hanau (DE)

(72) Inventors: Michael GOCK, Hanau (DE); Sabrina KRÄH, München (DE); Oliver TRAPP, München (DE)

(21) Appl. No.: 18/709,119

(22) PCT Filed: Oct. 17, 2022
(86) PCT No.: PCT/EP2022/078804

§ 371 Date: May 10, 2024

(30) Foreign Application Priority Data
Nov. 16, 2021 (EP) 21208544.3

(51) Int. Cl. B01J 31/24; B01J 37/16

(52) U.S. Cl. CPC B01J 31/2438; B01J 37/16; B01J 2231/4227; B01J 2231/4283; B01J 2531/824

(57) ABSTRACT

A phosphine of formula (1)

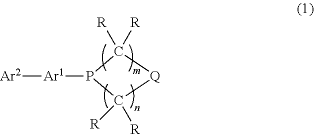

wherein
Q is SiR³R⁴, GeR³R⁴, SnR³R⁴, AsR³ or Se;
m is 0, 1, 2 or 3; n is 0, 1, 2 or 3; under the proviso that m+n≥1;
the groups R are, independently of each other, a hydrogen atom or a C₁₋₄ alkyl; or two groups R that bind to the same carbon atom together with this carbon atom form a 4- to 7-membered ring; or, under the proviso that m+n≥2, two groups R that bind to different carbon atoms together with these carbon atoms form a 4- to 7-membered ring;
Ar¹ and Ar² are aryls;
R³ and R⁴ are, independently of each other, a C₁₋₄ alkyl, a C₅₋₇ cycloalkyl or an aryl; or R³ and R⁴ together with the Si, Ge or Sn atom form a 4- to 7-membered ring.

The present invention relates to cyclic biaryl phosphines and to the use thereof as ligands in palladium complexes in palladium-catalyzed coupling reactions (such as the Buchwald-Hartwig coupling).

A cross coupling is understood to mean a coupling reaction in which two different molecules react with one another in the presence of a suitable catalyst to form a carbon-carbon or carbon-heteroatom bond. Palladium-catalyzed cross-couplings in particular are of great practical importance (e.g. in the synthesis of active pharmaceutical ingredients). By way of example, the Stille coupling, the Suzuki coupling (also known as the Suzuki-Miyaura coupling) or the Negishi coupling can be mentioned in this context. In 2010, R. F. Heck, E. Negishi, and A. Suzuki received the Nobel Prize in Chemistry for their work on coupling reactions.

The following reviews describe the use of cross-coupling reactions in the synthesis of active pharmaceutical ingredients:
H. C. Shen, “Selected Applications of Transition Metal-Catalyzed Carbon-Carbon Cross-Coupling Reactions in the Pharmaceutical Industry,” pp. 25-96, in “Applications of Transition Metal Catalysis in Drug Discovery and Development: An Industrial Perspective,” ed.: M. L. Crawley and B. M. Trost, 2012, John Wiley & Sons;
Q. Gu et al., “Palladium catalyzed C—C and C—N bond forming reactions: An update on the synthesis of pharmaceuticals from 2015-2020,” Org. Chem. Front., 2021, 8, pp. 384-414.

Another coupling reaction known to a person skilled in the art is the Buchwald-Hartwig coupling, in which an aryl or heteroaryl halide or pseudohalide and a primary or secondary amine are reacted with one another in the presence of a base and a palladium-containing catalyst to form a C—N bond.

The current state in the field of C—N coupling reactions is summarized in the following reviews:
R. Dorel et al., “The Buchwald-Hartwig Amination After 25 Years,” Angew. Chem. Int. Ed., 2019, 58, pp. 17118-17129;
S. L. Buchwald et al., “Dialkylbiaryl phosphines in Pd-catalyzed amination: a user's guide,” Chem. Sci., 2011, 2, pp. 27-50;
S. L. Buchwald et al., “Biaryl Phosphane Ligands in Palladium-Catalyzed Amination.” Angew. Chem. Int. Ed., 2008, 47, pp. 6338-6361;
S. L. Buchwald et al., “Applications of Palladium-Catalyzed C—N Cross-Coupling Reactions,” Chem. Rev., 2016 116, pp. 12564-12649.

Conventional catalysts in the field of coupling reactions are palladium complexes containing one or more phosphine ligands and optionally other ligands. These Pd complexes with suitable phosphine ligands can be prepared beforehand and stored until they are used, or alternatively generated in situ during the reaction to be catalyzed (e.g. by separate addition of a palladium salt and the phosphine to the reaction medium so that the formation of a phosphine-containing Pd complex takes place only in the reaction medium).

It is known that non-cyclic biaryl phosphines can act as ligands for Pd complexes in palladium-catalyzed coupling reactions (e.g. Buchwald-Hartwig coupling); see, for example, S. L. Buchwald et al., “Dialkylbiaryl phosphines in Pd-catalyzed amination: a user's guide,” Chem. Sci., 2011, 2, pp. 27-50. In these non-cyclic phosphines, there is no phosphorus-containing ring (i.e., no ring containing phosphorus as a ring atom).

Cyclic biaryl phosphines (i.e., phosphines in which the phosphorus atom is one of the ring-forming atoms) are also known as ligands for Pd complexes in palladium-catalyzed coupling reactions.

S. Shekhar et al., ACS Catal., 2019, 9, pp. 11691-11708, and S. Shekhar et al., ACS Catal., 2020, 10, pp. 15008-15018, describe biaryl phosphorinanes and their use as Pd complex ligands for palladium-catalyzed coupling reactions.

C. Maumela et al., RSC Adv., 2021, 11, pp. 26883-26891, describe biaryl phobanes and biaryl phosphatrioxa-adamantanes and their use as Pd complex ligands for palladium-catalyzed Suzuki couplings.

WO 2012/009698 A1 describes a monocyclic, bicyclic or tricyclic biaryl phosphine, wherein the heterocyclic ring system contains, in addition to the phosphorus atom, four carbon atoms and optionally at least one further ring atom selected from carbon, oxygen, nitrogen, phosphorus, and sulfur.

The formation of a carbon-heteroatom bond (in particular a C—N bond) via a coupling reaction, in particular a Buchwald-Hartwig coupling, requires relatively long reaction times when using N-heterocyclic aryl halides. If more efficient catalysts are not available, the reaction temperature can, for example, be increased in order to achieve shorter reaction times. However, this usually leads to undesired side reactions that further reduce the yield of the desired coupling product.

One object of the present invention is to provide suitable phosphines that can be used as ligands in palladium complexes in palladium-catalyzed cross-coupling reactions. In particular, these phosphines should also allow for efficient C—N coupling reactions (e.g. in the form of a Buchwald-Hartwig coupling) as ligands in palladium complexes when using N-heterocyclic aryl halides or pseudohalides as reactants.

The object is achieved by a phosphine of formula (1)

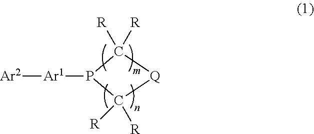

wherein
Q is SiR³R⁴, GeR³R⁴, SnR³R⁴, AsR³ or Se;
m is 0, 1, 2 or 3; n is 0, 1, 2 or 3; under the proviso that m+n≥1;
the groups R are, independently of each other, a hydrogen atom or a C₁₋₄ alkyl; or two groups R that bind to the same carbon atom, are in each case a divalent alkylene group and together with the carbon atom to which they are bound form a 4- to 7-membered (in particular 5- to 6-membered) ring; or, under the proviso that m+n≥2, two groups R that bind to different carbon atoms, are in each case a divalent alkylene group and together with the carbon atoms to which they are bound form a 4- to 7-membered (in particular 5- to 6-membered) ring;
Ar¹ is an aryl optionally substituted with one or more C₁₋₆ alkyl groups;
Ar² is an aryl optionally substituted with one or more C₁₋₆ alkyl groups;
R³ and R⁴ are, independently of each other, a C₁₋₄ alkyl, a C₅₋₇ cycloalkyl or an aryl (in particular a phenyl optionally substituted with one or more C₁₋₆ alkyl groups); or R³ and
R⁴ are in each case a divalent alkylene group and together with the Si, Ge or Sn atom form a 4- to 7-membered ring.

In addition to the phosphorus atom, the cyclic biaryl phosphines of the present invention contain a further heteroatom as a ring atom in the heterocyclic ring, wherein this additional hetero ring atom is Si, Ge, Sn, As or Se. As described in more detail below, the use of the phosphine according to the invention as a ligand in Pd complexes makes it possible to perform a C—C cross-coupling reaction or a C—N cross-coupling reaction, for example the Buchwald-Hartwig coupling, which leads to high yields at relatively low reaction temperatures and relatively short reaction times, even when an N-heterocyclic aryl halide or pseudohalide is used as a reactant.

The palladium complex containing the phosphine according to the invention as a ligand can already be prepared prior to the reaction to be catalyzed and can optionally be stored until it is used. Alternatively, it is also possible for the phosphine according to the invention and a palladium compound acting as a precursor to be added to the reaction medium so that the formation of a palladium complex containing the phosphine according to the invention as a ligand takes place in situ in the reaction medium of the coupling reaction.

If, in formula (1), two groups R that are bonded to the same carbon atom form a 4- to 7-membered ring together with this carbon atom, a spiro ring system is present.

If, in formula (1), two groups R that are bonded to two adjacent carbon atoms form a 4- to 7-membered ring together with these adjacent carbon atoms, a fused ring system is present.

If, in formula (1), two groups R that are bonded to two non-adjacent carbon atoms form a 4- to 7-membered ring together with these non-adjacent carbon atoms, a bridged ring system is present.

In a preferred embodiment, the following applies to the phosphine of formula (1):
Q is SiR³R⁴ or SnR³R⁴ (more preferably SiR³R⁴);
m is 1, 2 or 3; n is 1, 2 or 3; under the proviso that the following relationship is met: 3≤m+n≤5;
Ar¹ is a phenyl or naphthyl, wherein the phenyl or naphthyl is optionally substituted with one or more C₁₋₆ alkyl groups;
Ar² is a phenyl or naphthyl, wherein the phenyl or naphthyl is optionally substituted with one or more C₁₋₆ alkyl groups;
R³ and R⁴ are, independently of each other, a C₁₋₄ alkyl or a phenyl optionally substituted with one or more C₁₋₆ alkyl groups.

The phosphine according to the invention preferably has the following formula (1a):

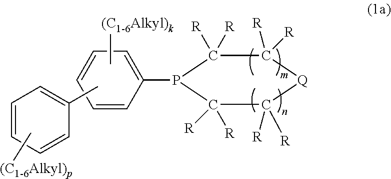

wherein
m is 0 or 1 (more preferably m=1) and n is 1 or 2 (more preferably n=1);
k is 0, 1, 2, 3 or 4;
p is 0, 1, 2, 3, 4, or 5;
Q and R each have the meaning given above.

The following preferably applies to formula (1a):

Q is SiR³R⁴ or SnR³R⁴, particularly preferably SiR³R⁴;
R³ and R⁴ are, independently of each other, a C₁₋₄ alkyl or a phenyl optionally substituted with one or more C₁₋₆ alkyl groups.

In a preferred embodiment of the present invention, the phosphine has the following formula (1b):

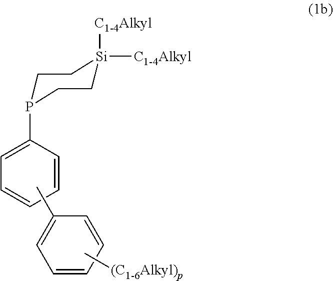

wherein
p is 0, 1, 2, 3, 4 or 5, more preferably 1, 2, 3 or 4.

The present invention also relates to a method for preparing the above-described phosphine according to the invention, wherein a phosphine of formula (5)

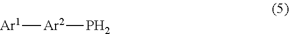

wherein
Ar¹ and Ar² each have the meaning described above;
   is at least partially deprotonated with a base and the resulting phosphide is reacted with a compound of formula (6)

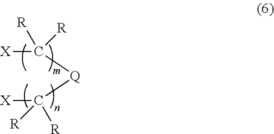

wherein
X is a halogen atom (e.g. Cl, Br or I),
Q, R, m and n each have the meaning described above.

Suitable bases with which a primary phosphine can be converted to a phosphide by deprotonation are known to a person skilled in the art. For example, the base is an organolithium compound (e.g. butyllithium).

The phosphine of formula (5) can be obtained, for example, by reducing a compound of formula (4)

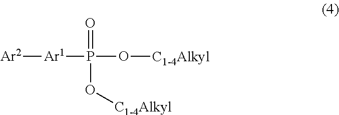

wherein
Ar¹ and Ar² each have the meaning described above;
   in the presence of a reducing agent to form the phosphine of formula (5).

The reducing agent is, for example, a metal hydride (e.g. lithium aluminum hydride).

When the phosphine of the above formula (1a) is prepared using the method according to the invention, the phosphide obtained after the deprotonation of the phosphine of formula (5) is reacted with a compound of formula (6a):

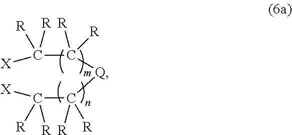

wherein
m is 0 or 1 (more preferably m=1) and n is 1 or 2 (more preferably n=1);
X is in each case a halogen atom (e.g. Cl, Br or I);
Q and R each have the meaning given above for formula (1a).

When the phosphine of the above formula (1b) is prepared using the method according to the invention, the phosphide obtained after the deprotonation of the phosphine of formula (5) is reacted with a compound of formula (6b):

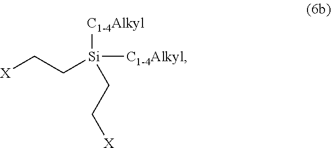

wherein X is in each case a halogen atom (e.g. Cl, Br or I).

The compound of formula (4) can be prepared, for example, by reacting a compound of formula (2)

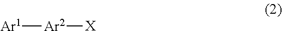

wherein
X is a halogen atom (e.g. Cl, Br or I),
Ar¹ and Ar² each have the meaning described above,
   with an organometallic compound, preferably an organolithium compound (e.g. butyllithium), to form an intermediate product, and by subsequently reacting the intermediate product with a compound of formula (3)

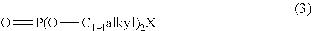

wherein
X is a halogen atom (e.g. Cl, Br or I).

The present invention also relates to a palladium complex containing the phosphine according to the invention described above as a ligand L¹.

The palladium complex can additionally have one or more ligands L² that are in each case not a phosphine according to the invention. Suitable ligands for palladium complexes are known to a person skilled in the art.

For example, the other ligands L² of the Pd complex according to the invention are selected independently of each other from a halide (e.g. Cl⁻, Br⁻ or I⁻); an aryl; a nitrile (e.g. acetonitrile, propionitrile or benzonitrile); a carboxylate (e.g. acetate); a conjugated dienone (e.g. a 1,4-dien-3-one such as dibenzylideneacetone (dba)); a phosphine other than a phosphine according to the invention (e.g. a non-cyclic phosphine); a pseudohalide (e.g. CN⁻ or OCN⁻); an amine or acetylacetonate.

The ligand L², which is not a phosphine according to the invention, can be a monodentate or alternatively a polydentate ligand. Examples of such polydentate ligands include arylalkylamines or arylamines (e.g. phenethylamine or naphthylamine) and monoanions thereof.

If the ligand L² is a phosphine, it is preferably a non-cyclic phosphine, i.e., a phosphine that does not have a phosphorus-containing ring. Suitable non-cyclic phosphine ligands for palladium complexes are known to a person skilled in the art.

The phosphine ligand L², which is not a phosphine according to the invention, is, for example, a tri-C₁₋₆ alkylphosphine, a tri-C₅₋₇ cycloalkylphosphine or a triarylphosphine (in particular a triphenylphosphine), wherein each of the aryl groups (which are preferably phenyl groups) is optionally substituted by one or more C₁₋₄ haloalkyl groups (e.g. —CF₃), C₁₋₄ alkyl groups (e.g. methyl) or C₁₋₄ alkoxy groups (e.g. methoxy).

For example, the phosphine ligand L² has one of the following formulas (7.1), (7.2) or (7.3):

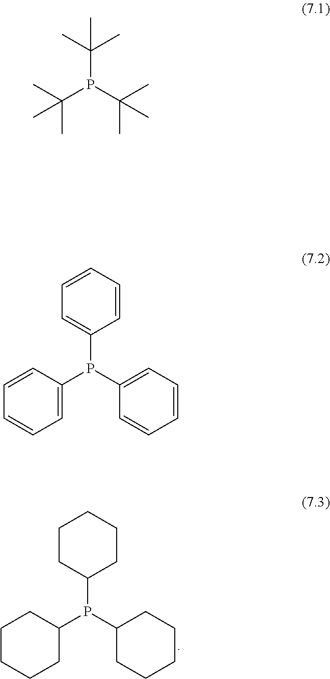 [SMILES: CC(C)(C)P(C(C)(C)C)C(C)(C)C ; c1ccc(P(c2ccccc2)c2ccccc2)cc1 ; C1CCC(P(C2CCCCC2)C2CCCCC2)CC1]

Alternatively, the phosphine ligand L² can be a phosphine of the following formula (8):

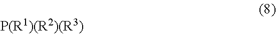

wherein
R¹ and R² are selected, independently of each other, from C₁₋₆ alkyl and C₅₋₇ cycloalkyl (e.g. cyclohexyl),
R³ is biphenyl optionally substituted by one or more C₁₋₆ alkyl groups, C₁₋₆ alkoxy groups, phenyl groups or pyridyl groups.

For example, the phosphine ligand L² has one of the following formulas (8.1), (8.2), (8.3) or (8.4):

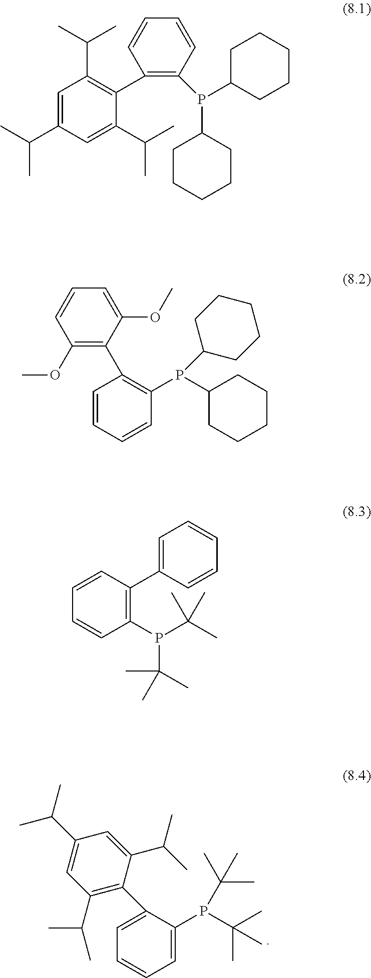 [SMILES: CC(C)c1cc(C(C)C)c(-c2ccccc2P(C2CCCCC2)C2CCCCC2)c(C(C)C)c1 ; COc1cccc(OC)c1-c1ccccc1P(C1CCCCC1)C1CCCCC1 ; CC(C)(C)P(c1ccccc1-c1ccccc1)C(C)(C)C ; CC(C)c1cc(C(C)C)c(-c2ccccc2P(C(C)(C)C)C(C)(C)C)c(C(C)C)c1]

For example, the palladium complex according to the invention has the following formula (10):

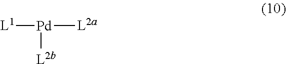

wherein
L¹ is a phosphine according to the invention,
L^{2a} and L^{2b} are in each case a ligand other than a phosphine according to the invention.

With regard to suitable ligands L^{2a} and L^{2b}, the above statements regarding ligand L² can be referred to. For example, the ligands L^{2a} and L^{2b} of the Pd complex according to the invention are selected independently of each other from a halide (e.g. Cl⁻, Br⁻ or I⁻); an aryl; a nitrile (e.g. acetonitrile, propionitrile or benzonitrile); a carboxylate (e.g. acetate); a conjugated dienone (e.g. a 1,4-dien-3-one such as dibenzylideneacetone (dba)); a phosphine other than a phosphine according to the invention (e.g. a non-cyclic phosphine); a pseudohalide (e.g. CN⁻ or OCN⁻); an amine or acetylacetonate.

An exemplary palladium complex of the present invention has the following formula (10.1):

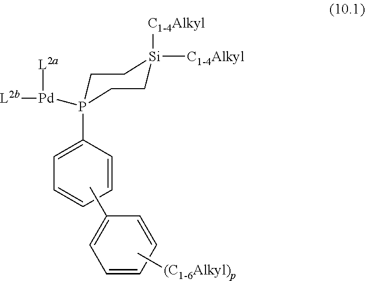

wherein the ligands L^{2a} and L^{2b} have the meanings given above and p is 0, 1, 2, 3 or 4.

For example, the ligand L^{2a} is dibenzylideneacetone (dba) or acetonitrile and the ligand L^{2b} is selected from one of the ligands indicated above for L².

A further exemplary palladium complex of the present invention has the following formula (10.2):

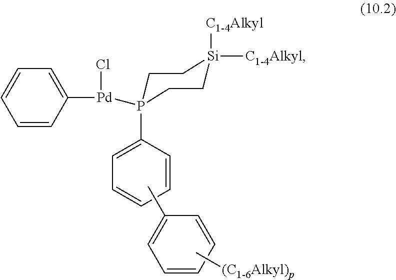

wherein p is 0, 1, 2, 3 or 4.

A further exemplary palladium complex of the present invention has the following formula (10.3):

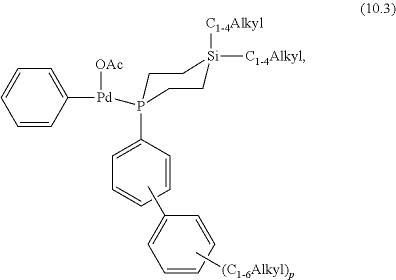

wherein p is 0, 1, 2, 3 or 4.

As already mentioned above, the phosphine according to the invention and a palladium compound acting as a precursor that does not already contain the phosphine according to the invention can be added to the reaction medium so that the formation of a palladium complex containing the phosphine according to the invention as a ligand takes place in situ in the reaction medium of the coupling reaction.

The present invention therefore also relates to a composition containing
a palladium compound, and
the phosphine according to the invention described above.

The palladium compound of the composition according to the invention usually does not contain any phosphine according to the invention.

Optionally, the composition can be present in the form of a kit containing the palladium compound and the phosphine according to the invention in separate containers.

The palladium compound is, for example, a palladium salt or a palladium complex, the ligands of which are not a phosphine according to the invention.

The palladium salt is, for example, a palladium acetate, a palladium halide (for example a palladium chloride, palladium bromide or palladium iodide) or a palladium pseudohalide or a mixture of at least two of these salts.

If the palladium compound is a palladium complex, the ligands thereof are, for example, selected independently of each other from a halide (e.g. Cl⁻, Br⁻ or I⁻); a phosphine other than a phosphine according to the invention (e.g. a non-cyclic phosphine); a conjugated dienone (e.g. a 1,4-dien-3-one such as dibenzylideneacetone (dba)); a polyene (for example a polyene with 2-4 double bonds, preferably a cyclic polyene, in particular a cyclic polyene with 2-4 double bonds, for example cyclooctadiene or cyclooctatetraene); a nitrile (e.g. acetonitrile, propionitrile or benzonitrile); an acetylacetonate; a carboxylate (e.g. acetate); a pseudohalide (e.g. CN⁻ or OCN⁻); an amine or an aryl.

With regard to the phosphine ligand that is not a phosphine according to the invention, reference can be made to the above statements regarding ligand L². Preferably, the phosphine ligand that is not a phosphine according to the invention is a non-cyclic phosphine, i.e., a phosphine that does not have a phosphorus-containing ring. Suitable non-cyclic phosphine ligands for palladium complexes are known to a person skilled in the art. The phosphine ligand, which is not a phosphine according to the invention, is, for example, a tri-C₁₋₆ alkylphosphine, a tri-C₅₋₇ cycloalkylphosphine or a triarylphosphine (in particular a triphenylphosphine), wherein each of the aryl groups (which are preferably phenyl groups) is optionally substituted by one or more C₁₋₄ haloalkyl groups (e.g. —CF₃), C₁₋₄ alkyl groups (e.g. methyl) or C₁₋₄ alkoxy groups (e.g. methoxy). Alternatively, the phosphine ligand that is not a phosphine according to the invention can be, for example, a phosphine of formula (8) described above.

The following can be mentioned as exemplary palladium compounds of the composition according to the invention: a palladium dibenzylidene complex (e.g. Pd₂ (dba)₃ or Pd (dba)₂); PdCl₂ (PR₃)₂, wherein R is a phenyl (optionally substituted with one or more C₁₋₆ alkyl groups), a C₅₋₇ cycloalkyl or a C₁₋₆ alkyl; a palladium acetate (e.g. Pd₂ (OAc)₃); a palladium acetylacetonate; a PdX₂, wherein X is a halide or pseudohalide; Pd(RCN)₂Cl₂, wherein R is a phenyl or methyl.

The present invention also relates to the use of the above-described palladium complex according to the invention or of the above-described composition according to the invention as a catalyst in a cross-coupling reaction.

The cross-coupling reaction is, for example, a C—C or C—N cross-coupling reaction.

A preferred C—N cross-coupling reaction is the Buchwald-Hartwig coupling. As is known to a person skilled in the art, the Buchwald-Hartwig coupling is a coupling reaction in which an aryl or heteroaryl halide, pseudohalide or sulfonate and a primary or secondary amine are reacted with one another in the presence of a palladium-containing catalyst (and preferably a base) to form a C—N bond.

The C—C cross-coupling reaction is, for example, a Suzuki-Miyaura coupling. As is known to a person skilled in the art, the Suzuki-Miyaura coupling is a coupling reaction in which an organoboron compound and, for example, an aryl or heteroaryl halide, pseudohalide or sulfonate are reacted with one another in the presence of a palladium-containing catalyst to form a C—C bond.

The present invention also relates to a method for preparing an arylamine or heteroarylamine, wherein a compound of formula (9)

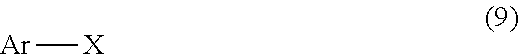

wherein
Ar is an aryl or heteroaryl,
X is a halogen atom, a sulfonate group (e.g. trifluoromethane sulfonate —O-Tf) or a
pseudohalogen group (e.g. —CN, —OCN or —NCO),
   is reacted with a primary or secondary amine in the presence of the above-described palladium complex according to the invention or the above-described composition according to the invention.

Suitable reaction conditions for the Buchwald-Hartwig coupling are known to a person skilled in the art. The reaction is preferably carried out in the presence of a base.

### EXAMPLES

### Preparation of a Cyclic Biaryl Phosphine According to the Invention

A cyclic biaryl phosphine according to the invention of the following formula (1)

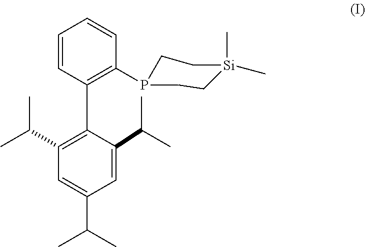

was prepared according to the following reaction scheme:

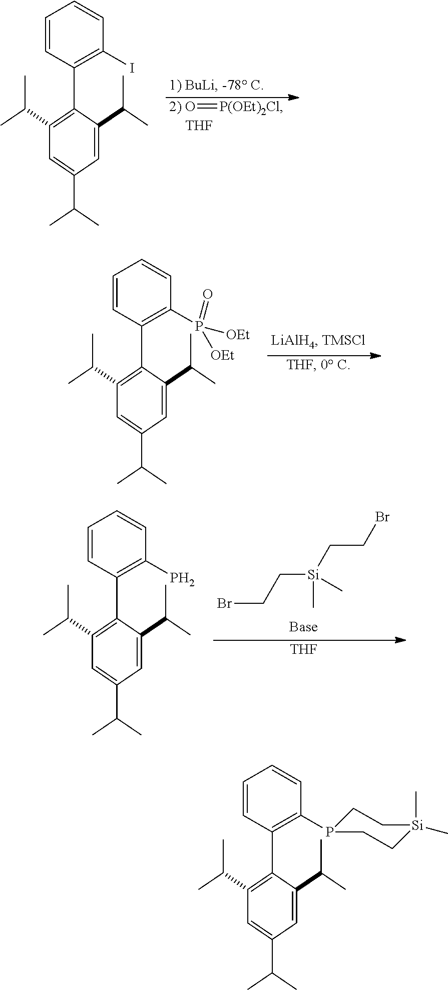

Chemical name of the cyclic biaryl phosphine of formula (1): 4,4-dimethyl-1-(2′,4′,6′-triisopropyl-[1,1′-biphenyl]-2-yl)-1,4-phosphasilinane

The preparation of the intermediate products

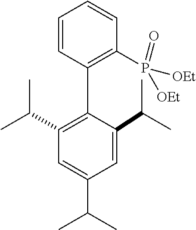

(chemical name: diethyl(2′,4′,6′-triisopropyl-[1,1′-biphenyl]-2-yl) phosphonate) and

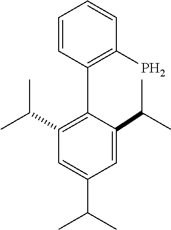

(chemical name: 2′,4′,6′-triisopropyl-[1,1′-biphenyl]-2-yl) phosphane) was carried out according to the syntheses described by S. Shekhar et al., ACS Catal., 2019, 9, pp. 11691-11708.

### Diethyl(2′,4′,6′-triisopropyl-[1,1′-biphenyl]-2-yl) phosphonate

2′-Iodo-2,4,6-triisopropyl-1,1′-biphenyl (438 mg; 1.08 mmol; 1.00 equiv.) under argon was dissolved in degassed, dry THF (5 mL), and n-BuLi (2.50 M in hexane; 0.47 mL; 1.10 mmol; 1.10 equiv.) was slowly added in drops at −78° C. for 30 minutes. The solution was stirred at −78° C. for 30 minutes. Diethylchlorophosphate (0.19 mL; 1.29 mmol; 1.20 equiv.) was added at −78° C., and the solution was stirred at −78° C. for another 30 minutes. The reaction solution was then stirred at room temperature for 5 hours. The reaction was quenched with an aqueous saturated solution of NH₄Cl (7 mL), filtered off, and the aqueous phase was extracted with EtOAc (3×7 mL). The combined organic phases were washed with a saturated sodium chloride solution (7 mL) and dried over Na₂SO₄. The solvent was removed in vacuo. The residue was purified by column chromatography (silica gel, hexane/EtOAc: 0-60% EtOAc). The product (335 mg; 0.80 mmol; 75%) was obtained as a colorless solid.

### (2′,4′,6′-triisopropyl-[1,1′-biphenyl]-2-yl) phosphane

LiAlH₄ (1.00 M in THF; 5.04 mL; 5.04 mmol; 3.00 equiv.) was added under argon at 0° C. to degassed, dry THF (5 mL). Trimethylchlorosilane (0.64 mL; 5.04 mmol; 3.00 equiv.) was added at 0° C. and the solution was stirred at 0° C. for 30 minutes. The solution was added in drops to a solution of diethyl(2′,4′,6′-triisopropyl-[1,1′-biphenyl]-2-yl) phosphonate (335 mg; 0.80 mmol; 1.00 equiv.) in THF (5 mL) at 0° C. The reaction solution was stirred overnight at room temperature, then cooled to 0° C. and quenched with EtOAc (10 mL) and an aqueous 1M HCl solution (26 mL). The reaction mixture was stirred for one hour and the aqueous phase was extracted with EtOAc (2×10 mL). The combined organic phases were washed with a saturated sodium chloride solution (10 mL), dried over NA₂SO₄ and the solvent was removed in vacuo. The product (239 mg; 0.76 mmol; 95%) was obtained as a colorless solid.

### 4,4-dimethyl-1-(2′,4′,6′-triisopropyl-[1,1′-biphenyl]-2-yl)-1,4-phosphasilinane

(2′,4′,6′-triisopropyl-[1,1′-biphenyl]-2-yl) phosphane (200 mg; 0.64 mmol; 1.00 equiv.) were dissolved in dry, degassed THF (3 mL). n-BuLi (2.5 M in hexane; 0.51 mL; 1.28 mmol; 2.00 equiv.) was added in drops at −78° C. The mixture was stirred at room temperature for 10 minutes. Bis(bromoethyl)dimethylsilane (175 mg; 0.64 mmol; 1.00 equiv.) was then added at −78° C. The reaction was stirred at room temperature for 4 hours. The reaction was quenched with a degassed, aqueous, saturated solution of NH₄Cl (5 mL). The aqueous phase was washed with degassed, dry EtOAc (3×7 mL) and the combined organic phases were dried over Na₂SO₄. The solvent was removed in vacuo. It was possible to isolate the product (259 mg; 0.61 mmol; 95%) as a colorless viscous liquid.

### Use of Compositions Containing a Biaryl Phosphine and a Palladium Compound as a Catalyst in a Buchwald-Hartwig Coupling

In Examples 1-13 according to the invention described below, the cyclic biaryl phosphine of formula (1) was used, i.e.:

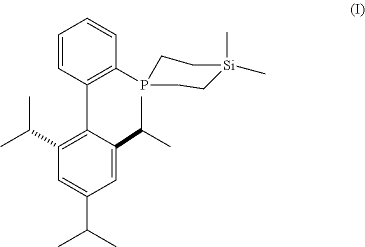

In Comparative Example 1, a non-cyclic biaryl phosphine of the following formula (II) was used:

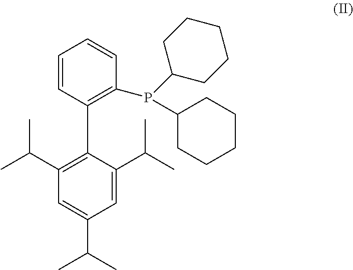 [SMILES: CC(C)c1cc(C(C)C)c(-c2ccccc2P(C2CCCCC2)C2CCCCC2)c(C(C)C)c1]

A palladium dibenzylidene complex (Pd₂ (dba)₃) was used as the palladium compound in all examples (i.e., Examples 1-13 according to the invention and Comparative Example 1). This palladium compound and the phosphine of formula (1) or (II) were added to the reaction medium so that a palladium complex containing the phosphine as one of its ligands could be formed in situ.

In Example 1, 2-chloroquinoline and piperidine were used as reactants for the Buchwald-Hartwig coupling.

### Example 1

2-chloroquinoline (150 mg; 0.92 mmol; 1.00 equiv.) was dissolved in dry, degassed toluene (6 mL). Pd₂ (dba)₃ (16.8 mg; 0.02 mmol; 0.02 equiv.), NaOtBu (123 mg; 1.28 mmol; 1.40 equiv.), the cyclic biaryl phosphine of formula (1) (0.5 M in toluene; 0.07 mL; 0.04 mmol; 0.04 equiv.) and piperidine (109 mL; 1.10 mmol; 1.20 equiv.) were added. The reaction was stirred at 60° C. for 2 hours and then quenched with an aqueous, saturated solution of NH₄Cl. The aqueous phase was washed with EtOAc (3×8 mL) and the combined organic phases were dried over Na₂SO₄. The solvent was removed in vacuo. The product was purified by column chromatography (silica gel, hexane/EtOAc: 20/1). It was possible to isolate 2-(piperidin-1-yl) quinoline (173 mg; 0.81 mmol; 89%) as a colorless solid.

In Examples 2-13 according to the invention, the reactants were varied (see Table 1 below), but the synthesis conditions were identical to the synthesis conditions used in Example 1.

In Comparative Example 1, the reactants and the synthesis conditions were identical to the reactants and synthesis conditions used in Example 1. However, instead of the phosphine of formula (1) according to the invention, the phosphine of formula (II) was used.

The results of Examples 1-13 according to the invention are summarized in Table 1 below.

TABLE 1
Amines used as reactants, reaction products and product yields obtained in the
Buchwald-Hartwig coupling in Examples 1-13 according to the invention

| Example | Heteroaryl | Amine | Reaction product | Yield [%] |
|---|---|---|---|---|
| 1 | 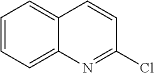 [SMILES: Clc1ccc2ccccc2n1] | 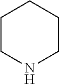 | 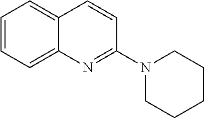 [SMILES: c1ccc2nc(N3CCCCC3)ccc2c1] | 88 |
| 2 | 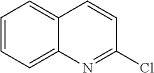 [SMILES: Clc1ccc2ccccc2n1] | 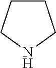 | 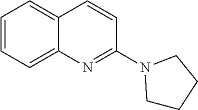 [SMILES: c1ccc2nc(N3CCCC3)ccc2c1] | 74 |
| 3 | 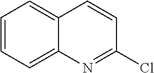 [SMILES: Clc1ccc2ccccc2n1] | 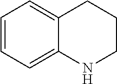 | 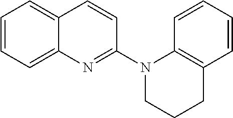 [SMILES: c1ccc2c(c1)CCCN2c1ccc2ccccc2n1] | 100 |
| 4 | 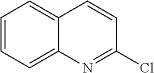 [SMILES: Clc1ccc2ccccc2n1] | 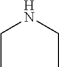 | 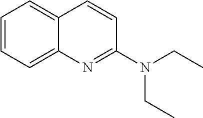 | 42 |
| 5 | 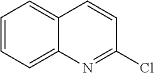 [SMILES: Clc1ccc2ccccc2n1] | 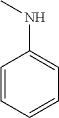 | 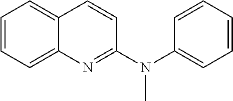 [SMILES: CN(c1ccccc1)c1ccc2ccccc2n1] | 86 |
| 6 | 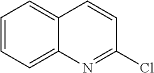 [SMILES: Clc1ccc2ccccc2n1] | 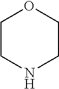 | 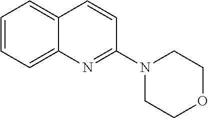 [SMILES: c1ccc2nc(N3CCOCC3)ccc2c1] | 95 |
| 7 | 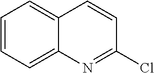 [SMILES: Clc1ccc2ccccc2n1] | 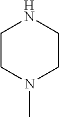 | 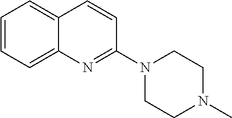 [SMILES: CN1CCN(c2ccc3ccccc3n2)CC1] | 67 |
| 8 | 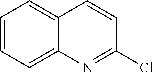 [SMILES: Clc1ccc2ccccc2n1] | 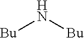 | 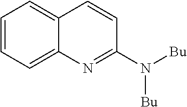 | 79 |
| 9 | 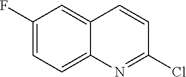 [SMILES: Fc1ccc2nc(Cl)ccc2c1] | 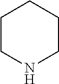 | 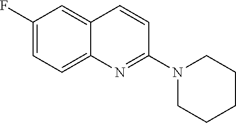 [SMILES: Fc1ccc2nc(N3CCCCC3)ccc2c1] | 82 |
| 10 | 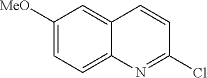 [SMILES: COc1ccc2nc(Cl)ccc2c1] | 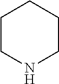 | 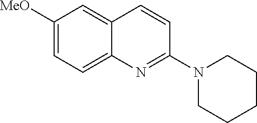 [SMILES: COc1ccc2nc(N3CCCCC3)ccc2c1] | 100 |
| 11 | 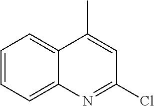 | 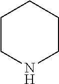 | 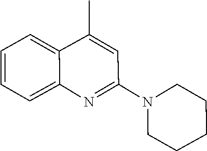 [SMILES: Cc1cc(N2CCCCC2)nc2ccccc12] | 98 |
| 12 | 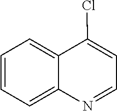 [SMILES: Clc1ccnc2ccccc12] | 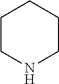 | 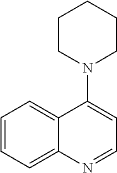 [SMILES: c1ccc2c(N3CCCCC3)ccnc2c1] | 40 |
| 13 | 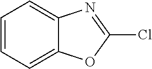 | 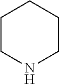 | 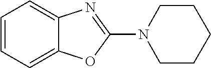 [SMILES: c1ccc2oc(N3CCCCC3)nc2c1] | 86 |

Although in all examples one of the reactants was an N-heterocyclic aryl halide and a very short reaction time was selected at a relatively mild reaction temperature, the use of the cyclic biaryl phosphine according to the invention as a ligand of a palladium complex in a palladium-catalyzed Buchwald-Hartwig coupling resulted in high product yields.

The result of Comparative Example 1 is shown in Table 2 below.

TABLE 2
Amines used as reactants and product yield in the Buchwald-Hartwig coupling in
Comparative Example 1

| | | | Desired reaction | |
|---|---|---|---|---|
| | Heteroaryl | Amine | product | Yield |
| Comp. ex. 1 | 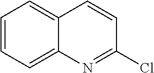 [SMILES: Clc1ccc2ccccc2n1] | 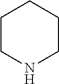 | 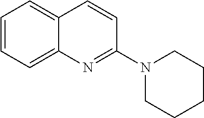 [SMILES: c1ccc2nc(N3CCCCC3)ccc2c1] | 0% |

### Use of a Composition Containing a Cyclic Biaryl Phosphine According to the Invention and a Palladium Compound as a Catalyst in a Suzuki-Miyaura Coupling

Using a cyclic biaryl phosphine of formula (1) according to the invention, a Suzuki-Miyaura coupling was carried out in Examples 14-17 according to the invention with the reactants and reaction conditions indicated in the following reaction scheme and in Table 3. The yields are shown in Table 3.

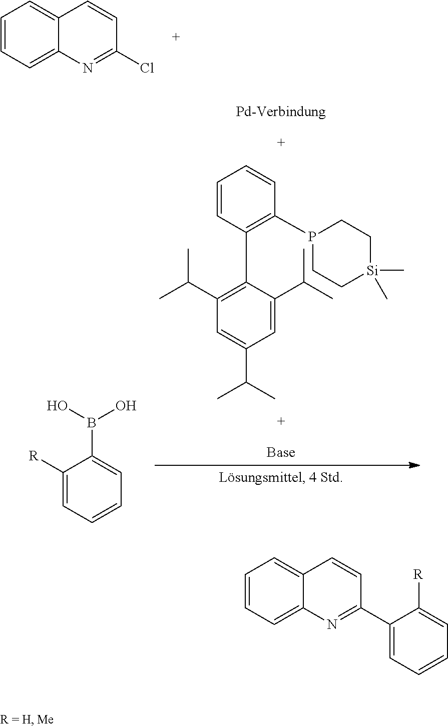

In Example 14, the preparation of 2-(o-tolyl) quinoline (i.e., R=Me) was as follows:

2-chloroquinoline (150 mg; 0.92 mmol; 1.00 equiv.) was dissolved in dry, degassed THF (5 mL) and degassed water (1 mL). Pd₂ (dba)₃ (16.8 mg; 0.02 mmol; 0.02 equiv.), CsOH×H₂O (216 mg; 1.28 mmol; 1.40 equiv.), the cyclic biaryl phosphine of formula (1)

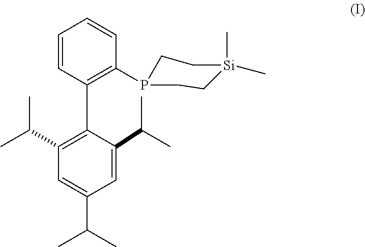

(0.5 M in toluene; 0.07 mL; 0.04 mmol; 0.04 equiv.), and o-tolylboronic acid (150 mg; 1.10 mmol; 1.20 equiv.) were added. The reaction was stirred at 60° C. for 4 hours and then quenched with an aqueous, saturated solution of NH₄Cl. The aqueous phase was washed with EtOAc (3×8 mL) and the combined organic phases were dried over Na₂SO₄. The solvent was removed in vacuo. The product was purified by column chromatography (silica gel, hexane/EtOAc: 10/1). It was possible to isolate 2-(o-tolyl) quinoline (179 mg; 0.82 mmol; 89%) as a colorless solid.

The Suzuki-Miyaura coupling in Examples 15-17 according to the invention was based on Example 14, but with the variations of the synthesis conditions shown in Table 3.

In each of Examples 14-17, the cyclic biaryl phosphine of formula (1) and a palladium compound (Pd₂ (dba)₃) or Pd(OAc)₂) were added to the reaction medium so that a palladium complex containing the cyclic biaryl phosphine according to the invention as one of its ligands could be formed in situ.

TABLE 3
Reaction yields in Examples 14-17

| | Pd | | | | Reaction | |
|---|---|---|---|---|---|---|
| | compound | R | Base | Solvent | temperature | Yield |
| Example | Pd₂(dba)₃ | Me | CsOH × | THF/H₂O | 60° C. | 89% |
| 14 | | | H₂O | | | |
| Example | Pd₂(dba)₃ | H | NaOtBu | THF/H₂O | Room | 35% |
| 15 | | | | | temperature | |
| Example | Pd₂(dba)₃ | H | CsOH × | THF/H₂O | Room | 54% |
| 16 | | | H₂O | | temperature | |
| Example | Pd(OAc)₂ | H | CsOH × | THF/H₂O | Room | 46% |
| 17 | | | H₂O | | temperature | |

Although in all examples one of the reactants was an N-heterocyclic aryl halide and a very short reaction time (4 hours) was selected at a mild reaction temperature, the use of the cyclic biaryl phosphine according to the invention as a ligand of a palladium complex in a palladium-catalyzed Suzuki-Miyaura coupling resulted in high product yields.

1. A phosphine of formula (1) 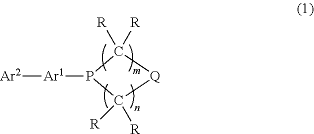
wherein
Q is SiR³R⁴, GeR³R⁴, SnR³R⁴, AsR³ or Se;
m is 0, 1, 2 or 3; n is 0, 1, 2 or 3; under the proviso that m+n≥1;
the groups R are, independently of each other, a hydrogen atom or a C₁₋₄ alkyl; or two groups R that bind to the same carbon atom are in each case a divalent alkylene group and together with the carbon atom to which they are bound form a 4- to 7-membered ring; or, under the proviso that m+n≥2, two groups R that bind to different carbon atoms are in each case a divalent alkylene group and together with the carbon atoms to which they are bound form a 4- to 7-membered ring;
Ar¹ is an aryl optionally substituted with one or more C₁₋₆ alkyl groups;
Ar² is an aryl optionally substituted with one or more C₁₋₆ alkyl groups;
R³ and R⁴ are, independently of each other, a C₁₋₄ alkyl, a C₅₋₇ cycloalkyl or an aryl; or R³ and R⁴ are in each case a divalent alkylene group and together with the Si, Ge or Sn atom form a 4- to 7-membered ring.

2. The phosphine according to claim 1, wherein
Q is SiR³R⁴ or SnR³R⁴, more preferably SiR³R⁴;
m is 1, 2 or 3; n is 1, 2 or 3; under the proviso that the following relationship is met: 3≤m+n≤5;
Ar¹ is a phenyl or naphthyl optionally substituted with one or more C₁₋₆ alkyl groups;
Ar² is a phenyl or naphthyl optionally substituted with one or more C₁₋₆ alkyl groups;
R³ and R⁴ are, independently of each other, a C₁₋₄ alkyl or a phenyl optionally substituted with one or more C₁₋₆ alkyl groups.

3. The phosphine according to claim 1, having the following formula (1a): 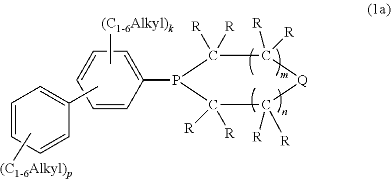
wherein
m is 0 or 1 (more preferably m=1) and n is 1 or 2 (more preferably n=1);
k is 0, 1, 2, 3 or 4;
p is 0, 1, 2, 3, 4, or 5;
Q and R.

4. The phosphine according to claim 1, having the following formula (1b): 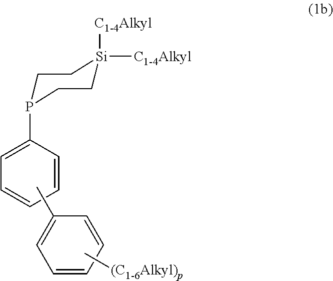
wherein
p is 0, 1, 2, 3, 4 or 5, more preferably 1, 2, 3 or 4.

5. A method for preparing the phosphine according to claim 1, wherein
a phosphine of formula (5) 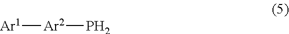
wherein
Ar¹ and Ar² are aryls optionally substituted with one or more C₁₋₆ alkyl
groups,
is at least partially deprotonated with a base and the resulting phosphide is reacted with a compound of formula (6) 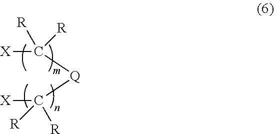
wherein
X is a halogen atom,
Q, R, m and n as indicated in said formula (1).

6. The method according to claim 5, wherein the phosphine of formula (5) is prepared by reducing a compound of formula (4) 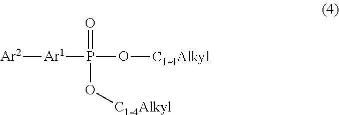
wherein
Ar¹ and Ar² are aryls optionally substituted with one or more C₁₋₆ alkyl
groups;
in the presence of a reducing agent.

7. The method according to claim 5, wherein the compound of formula (4) is prepared by reacting a compound of formula (2) 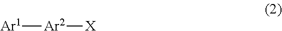
wherein
X is a halogen atom,
Ar¹ and Ar² are aryls optionally substituted with one or more C₁₋₆ alkyl groups,
with an organometallic compound, preferably an organolithium compound, to form an intermediate product, and by subsequently reacting the intermediate product with a compound of formula (3) 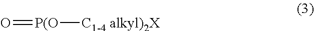
wherein X is a halogen atom.

8. A palladium complex containing the phosphine according to claim 1 as a ligand L¹.

9. The palladium complex according to claim 8, additionally containing one or more ligands L², each of which is not a phosphine according to said formula (1).

10. The palladium complex according to claim 9, wherein the ligands L² are selected, independently of each other, from a halide; an aryl; a nitrile; a carboxylate, a conjugated dienone; a phosphine which is not a phosphine of said formula (1); an amine or acetylacetonate.

11. A composition containing
a palladium compound, and
the phosphine according to claim 1.

12. The composition according to claim 11, wherein the palladium compound is a palladium salt or a palladium complex that does not contain a phosphine of said formula (1).

13. The composition according to claim 11, wherein the palladium salt is a palladium acetate, a palladium halide, a palladium pseudohalide, or a mixture of at least two of these salts.

14. The composition according to claim 12, wherein the ligands of the palladium complex are selected independently of each other from a halide; a phosphine which is not a phosphine of said formula (1); a conjugated dienone (preferably a 1,4-dien-3-one such as dibenzylideneacetone (dba)); a polyene (preferably a cyclic polyene with 2-4 double bonds such as cyclooctadiene or cyclooctatetraene); a nitrile; an acetylacetonate; a carboxylate; a pseudohalide; an amine or an aryl.

15. A use of the palladium complex according to claim 8 as a catalyst in a cross-coupling reaction.

16. The use according to claim 15, wherein the cross-coupling reaction is a C—C or a C—N cross-coupling reaction, in particular a Buchwald-Hartwig coupling.

17. A method for preparing an arylamine or heteroarylamine, wherein a compound of formula (9) 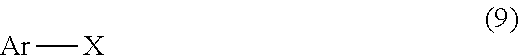
wherein
Ar is an aryl or heteroaryl,
X is a halogen atom, a sulfonate group, or a pseudohalogen group,
is reacted with a primary or secondary amine in the presence of the palladium complex according to claim 8.
